(19) United States
(12) Patent Application Publication
(10) Pub. No.: US 2010/0279994 A1
(43) Pub. Date: Nov. 4, 2010

(54) Ansamycin Formulations and Methods of Use Thereof

(75) Inventors: Jeffrey K. Tong, Brighton, MA (US); James R. Porter, Rowley, MA (US)

Correspondence Address: Foley Hoag, LLP (w/IPX), 155 Seaport Blvd., Boston, MA, 02210, US

(73) Assignee: INIFINITY DISCOVERY, INC., CAMBRIDGE, MA (US)

(21) Appl. No.: 11/993,097

(22) PCT Filed: Jun. 21, 2006
(86) PCT No.: PCT/US06/23987

§ 371 Date: Jun. 29, 2010

Related U.S. Application Data
(60) Provisional application No. 60/692,423, filed on Jun. 21, 2005.

(51) Int. Cl. A61K 31/395; A61P 35/00; A61P 31/12; A61P 37/02; A61P 1/16; A61P 9/00; A61P 29/00

(52) U.S. Cl. 514/183

(57) ABSTRACT

The present invention provides pharmaceutical compositions of reduced forms of benzoquinone-containing ansamycins, and salts thereof. The present invention also relates to the use of said pharmaceutical compositions in methods of treating and modulating disorders associated with hyperproliferation, such as cancer.

### BACKGROUND OF THE INVENTION

Heat Shock Protein 90 (“HSP90”) is a highly abundant protein which is essential for cell viability and which exhibits dual chaperon functions (J. Cell Biol. (2001) 154:267-273, Trends Biochem. Sci. (1999) 24:136-141). It plays a key role in the cellular stress-response by interacting with many proteins after their native conformation has been altered by various environmental stresses, such as heat shock, ensuring adequate protein-folding and preventing non-specific aggregation (Pharmacological Rev. (1998) 50:493-513). In addition, recent results suggest that HSP90 may also play a role in buffering against the effects of mutation, presumably by correcting the inappropriate folding of mutant proteins (Nature (1998) 396:336-342). However, HSP90 also has an important regulatory role under normal physiological conditions and is responsible for the conformational stability and maturation of a number of specific client proteins, of which about 40 are known (see. Expert. Opin. Biol Ther. (2002) 2(1): 3-24).

HSP90 antagonists are currently being explored in a large number of biological contexts where a therapeutic effect can be obtained for a condition or disorder by inhibiting one or more aspects of HSP90 activity. Although the primary focus has been on proliferative disorders, such as cancers, the use of HSP90 antagonists to treat other conditions is being explored. Examples of such conditions include viral disorders, inflammation, autoimmune disorders, stroke, ischemia, cardiac disorders, fibrogenetic disorders, scleroderma, polymyositis, systemic lupus, rheumatoid arthritis, liver cirrhosis, keloid formation, interstitial nephritis, and pulmonary fibrosis.

Geldanamycin is a macrocyclic lactam that is a member of the benzoquinone-containing ansamycin family of natural products. Geldanamycin's nanomolar potency and apparent selectivity for killing tumor cells, as well as the discovery that its primary target in mammalian cells is HSP90, has stimulated interest in its development as an anti-cancer drug. However, its extremely low solubility and the association of hepatotoxicity with the administration of geldanamycin has led to difficulties in developing an approvable agent for therapeutic applications. In particular, geldanamycin has poor water solubility, making it difficult to deliver in therapeutically effective doses.

### SUMMARY OF THE INVENTION

The present invention provides pharmaceutical compositions of hydroquinone analogs of amsamycins. The present invention also provides methods for the use of these pharmaceutical compositions in the treatment of diseases or conditions characterized by undesired cellular hyperproliferation, such as cancers, as well as other conditions and disorders associated with unwanted HSP90 activity or in which HSP90 plays a role in the cells involved in causing the disorder.

In one embodiment, the present invention provides a pharmaceutical composition comprising at least one pharmaceutically acceptable excipient and a compound of formula 1:

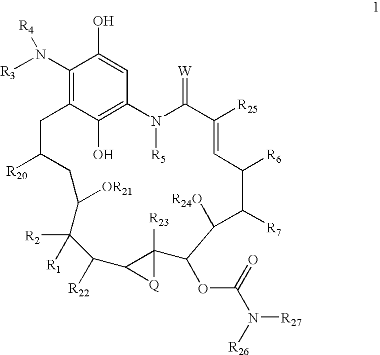

wherein independently for each occurrence:

W is oxygen or sulfur;

Q is oxygen, NR, N(acyl) or a bond;

R for each occurrence is independently selected from the group consisting of hydrogen, alkyl, cycloalkyl, heterocycloalkyl, aralkyl, heteroaryl, and heteroaralkyl;

R₁ is hydroxyl, alkoxyl, —OC(O)R₈, —OC(O)OR₉, —OC(O)NR₁₀R₁₁, —OSO₂R₁₂, —OC(O)NHSO₂NR₁₃R₁₄, —NR₁₃R₁₄, or halide;

R₂ is hydrogen, alkyl, or aralkyl; or R₁ and R₂ taken together, along with the carbon to which they are bonded, are —(C═O)—, —(C═N—OR)—, —(C═N—NHR)—, or —(C═N—R)—;

R₃ and R₄ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆; or R₃ taken together with R₄ represent a 4-8 membered optionally substituted heterocyclic ring;

R₅ is selected from the group consisting of H, alkyl, aralkyl, and a group having the formula 1a:

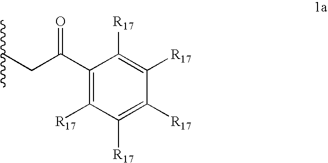

wherein R₁₇ is selected independently from the group consisting of hydrogen, halide, hydroxyl, alkoxyl, acyloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)NR₁₈)(R₁₉), and —CH₂O-heterocyclyl;

R₆ and R₇ are both hydrogen; or R₆ and R₇ taken together form a bond;

R₈ is hydrogen, alkyl, alkenyl, alkynyl, aryl, cycloalkyl, heterocycloalkyl, aralkyl, heteroaryl, heteroaralkyl, or —[(CR₂)ₚ]—R₁₆;

R₉ is alkyl, alkenyl, alkynyl, aryl, cycloalkyl, heterocycloalkyl, aralkyl, heteroaryl, heteroaralkyl, or —[(CR₂)ₚ]—R₁₆;

R₁₀ and R₁₁ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆; or R₁₀ and R₁₁ taken together with the nitrogen to which they are bonded represent a 4-8 membered optionally substituted heterocyclic ring;

R₁₂ is alkyl, alkenyl, alkynyl, aryl, cycloalkyl, heterocycloalkyl, aralkyl, heteroaryl, heteroaralkyl, or —[(CR₂)ₚ]—R₁₆;

R₁₃ and R₁₄ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆; or R₁₃ and R₁₄ taken together with the nitrogen to which they are bonded represent a 4-8 membered optionally substituted heterocyclic ring;

R₁₆ for each occurrence is independently selected from the group consisting of hydrogen, hydroxyl, acylamino, —N(R₁₈)COR₁₉, —N(R₁₈)C(O)OR₁₉, —N(R₁₈)SO₂(R₁₉), —CON(R₁₈)(R₁₉), C(O)N(R₁₈)(R₁₉), —SO₂N(R₁₈)(R₁₉), —N(R₁₈)(R₁₉), —OC(O)OR₁₈, —COOR₁₈, —C(O)N(OH)(R₁₈), —OS(O)₂OR₁₈, —S(O)₂OR₁₈, —OP(O)(OR₁₈)(OR₁₉), —N(R₁₈)P(O)(OR₁₈)(OR₁₉), and —P(O)(OR₁₈)(OR₁₉);

p is 1, 2, 3, 4, 5, or 6;

R₁₈ for each occurrence is independently selected from the group consisting of hydrogen, alkyl, aryl, cycloalkyl, heterocycloalkyl, aralkyl, heteroaryl, and heteroaralkyl;

R₁₉ for each occurrence is independently selected from the group consisting of hydrogen, alkyl, aryl, cycloalkyl, heterocycloalkyl, aralkyl, heteroaryl, and heteroaralkyl; or

R₁₈ taken together with R₁₉ represent a 4-8 membered optionally substituted ring;

R₂₀, R₂₁, R₂₂, R₂₄, and R₂₅, for each occurrence are independently alkyl;

R₂₃ is alkyl, —CH₂OH, —CHO, —COOR₁₈, or —CH(OR₁₈)₂;

R₂₆ and R₂₇ for each occurrence are independently selected from the group consisting of hydrogen, alkyl, aryl, cycloalkyl, heterocycloalkyl, aralkyl, heteroaryl, and heteroaralkyl;

the absolute stereochemistry at a stereogenic center of formula 1 may be R or S or a mixture thereof and the stereochemistry of a double bond may be E or Z or a mixture thereof.

In certain embodiments, the compound of formula 1 is selected from the group consisting of

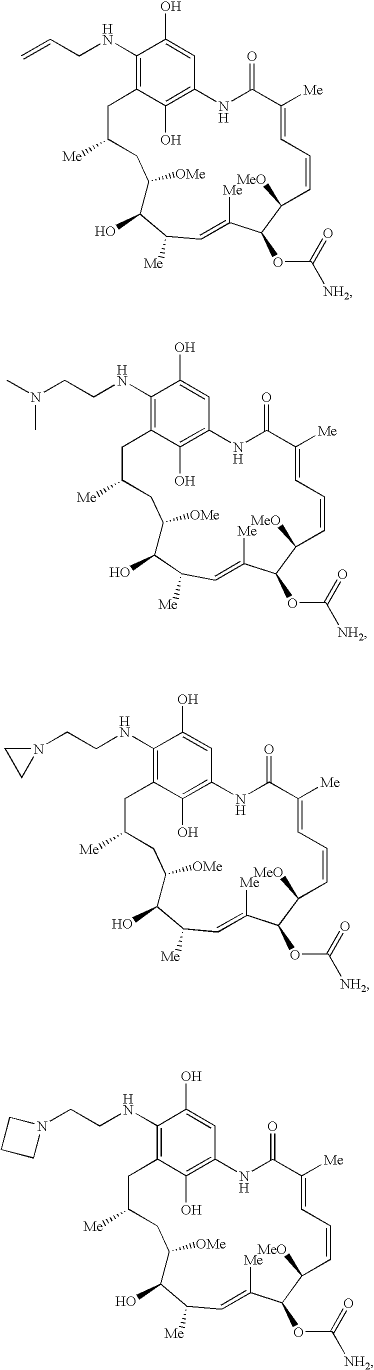

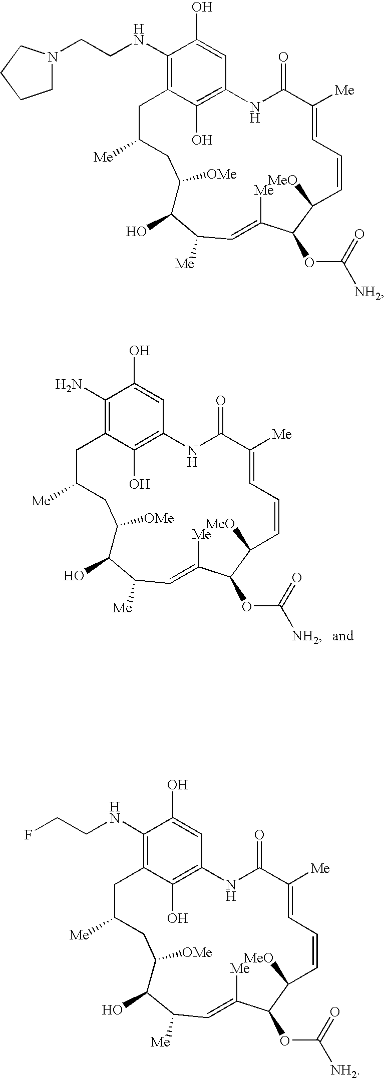

In certain embodiments, the pharmaceutical compositions described above further comprise an antioxidant and/or a metal chelator.

The antioxidant can be, e.g., ascorbate, cysteine hydrochloride, sodium bisulfate, sodium metabisulfite, sodium sulfite, thioglycerol, sodium mercaptoacetate, sodium formaldehyde sulfoxylate, ascorbyl palmitate, butylated hydroxyanisole, butylated hydroxytoluene, lecithin, propyl gallate, or alpha-tocopherol.

The metal chelator can be, e.g., citric acid, ethylenediamine tetraacetic acid (EDTA) or a salt thereof, DTPA (diethylene-triamine-penta-acetic acid) or a salt thereof, EGTA or a salt thereof, NTA (nitriloacetic acid) or a salt thereof, sorbitol or a salt thereof, tartaric acid or a salt thereof, N-hydroxy iminodiacetate or a salt thereof, hydroxyethyl-ethylene diamine-tetraacetic acid or a salt thereof, 1-propanediamine tetra acetic acid or a salt thereof, 3-propanediamine tetra acetic acid or a salt thereof, 1-diamino-2-hydroxy propane tetra-acetic acid or a salt thereof, 3-diamino-2-hydroxy propane tetra-acetic acid or a salt thereof, sodium gluconate, hydroxy ethane diphosphonic acid or a salt thereof, or phosphoric acid or a salt thereof.

### DETAILED DESCRIPTION OF THE INVENTION

The definitions of terms used herein are meant to incorporate the present state-of-the-art definitions recognized for each term in the chemical and pharmaceutical fields. Where appropriate, exemplification is provided. The definitions apply to the terms as they are used throughout this specification, unless otherwise limited in specific instances, either individually or as part of a larger group.

Where stereochemistry is not specifically indicated, all stereoisomers of the inventive compounds are included within the scope of the invention, as pure compounds as well as mixtures thereof. Unless otherwise indicated, individual enantiomers, diastereomers, geometrical isomers, and combinations and mixtures thereof are all encompassed by the present invention. Polymorphic crystalline forms and solvates are also encompassed within the scope of this invention.

The term “heteroatom” is art-recognized and refers to an atom of any element other than carbon or hydrogen. Illustrative heteroatoms include boron, nitrogen, oxygen, phosphorus, sulfur and selenium.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

The term “aryl” is art-recognized and refers to 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms “ortho”, “meta” and “para” are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl”, “heteroaryl”, or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “optionally substituted” refers to a chemical group, such as alkyl, cycloalkyl aryl, and the like, wherein one or more hydrogen may be replaced with a with a substituent as described herein, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on 560 of “Advanced Inorganic Chemistry” by Cotton and Wilkinson.

The terms “amine” and “amino” are art-recognized and refer to both unsubstituted and substituted amines, e.g., a moiety that may be represented by the general formulas:

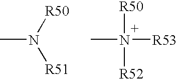

wherein R50, R51 and R52 each independently represent a hydrogen, an alkyl, an alkenyl, —(CH₂)ₘ-R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In other embodiments, R50 and R51 (and optionally R52) each independently represent a hydrogen, an alkyl, an alkenyl, or —(CH₂)ₘ-R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula:

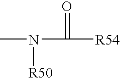

wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula:

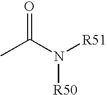

wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carboxyl” is art recognized and includes such moieties as may be represented by the general formulas:

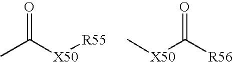

wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The term “carbamoyl” refers to —O(C═O)NRR′, where R and R′ are independently H, aliphatic groups, aryl groups or heteroaryl groups.

The term “oxo” refers to a carbonyl oxygen (═O).

The terms “oxime” and “oxime ether” are art-recognized and refer to moieties that may be represented by the general formula:

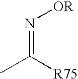

wherein R75 is hydrogen, alkyl, cycloalkyl, alkenyl, alkynyl, aryl, aralkyl, or —(CH₂)ₘ—R61. The moiety is an “oxime” when R is H; and it is an “oxime ether” when R is alkyl, cycloalkyl, alkenyl, alkynyl, aryl, aralkyl, or —(CH₂)ₘ—R61.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula:

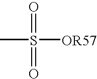

in which R57 is an electron pair, hydrogen, alkyl, cycloalkyl, or aryl.

The term “sulfate” is art recognized and includes a moiety that may be represented by the general formula:

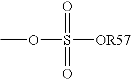

in which R57 is as defined above.

The term “sulfonamido” is art recognized and includes a moiety that may be represented by the general formula:

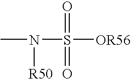

in which R50 and R56 are as defined above.

The term “sulfamoyl” is art-recognized and refers to a moiety that may be represented by the general formula:

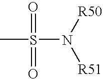

in which R50 and R51 are as defined above.

The term “sulfonyl” is art-recognized and refers to a moiety that may be represented by the general formula:

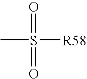

in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula:

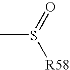

in which R58 is defined above.

The definition of each expression, e.g. alkyl, m, n, and the like, when it occurs more than once in any structure, is intended to be independent of its definition elsewhere in the same structure.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). Protected forms of the inventive compounds are included within the scope of this invention.

The term “composition” is intended to encompass a product comprising the specified ingredients in the specified amounts, as well as any product that results, directly or indirectly, from combinations of the specified ingredients in the specified amounts.

The term “HSP90 mediated disorder” or “disorder mediated by cells expressing HSP90” refers to pathological and disease conditions in which HSP90 plays a role. Such roles can be directly related to the pathological condition or can be indirectly related to the condition. The common feature to this class of conditions is that the condition can be ameliorated by inhibiting the activity, function, or association with other proteins of HSP90.

The term “pharmaceutically acceptable carrier” refers to a medium that is used to prepare a desired dosage form of a compound. A pharmaceutically acceptable carrier can include one or more solvents, diluents, or other liquid vehicles; dispersion or suspension aids; surface active agents; isotonic agents; thickening or emulsifying agents; preservatives; solid binders; lubricants; and the like. Remington's Pharmaceutical Sciences, Fifteenth Edition, E. W. Martin (Mack Publishing Co., Easton, Pa., 1975) and Handbook of Pharmaceutical Excipients, Third Edition, A. H. Kibbe ed. (American Pharmaceutical Assoc. 2000), disclose various carriers used in formulating pharmaceutical compositions and known techniques for the preparation thereof.

The compositions of the invention may contain an antioxidant. Pharmaceutically-acceptable antioxidants include, but are not limited to: (1) water soluble antioxidants, such as ascorbic acid, cysteine hydrochloride, sodium bisulfate, sodium metabisulfite, sodium sulfite, thioglycerol, sodium mercaptoacetate, and sodium formaldehyde sulfoxylate; (2) oil-soluble antioxidants, such as ascorbyl palmitate, butylated hydroxyanisole (BHA), butylated hydroxytoluene (BHT), lecithin, propyl gallate, alpha-tocopherol.

Compounds of formula 1 may oxidize on prolonged standing in solution or in solid form. Heavy metals, such as iron and copper, are capable of catalyzing oxidation reactions and can be found in trace quantities in typical reagents and labware. Protection from the oxidizing nature of heavy metals can be afforded by metal chelators.

Pharmaceutically-acceptable metal chelating agents that may be present in the compositions include, but are not limited to, citric acid, ethylenediamine tetraacetic acid (EDTA) or a salt thereof, DTPA (diethylene-triamine-penta-acetic acid) or a salt thereof, EGTA or a salt thereof, NTA (nitriloacetic acid) or a salt thereof, sorbitol or a salt thereof, tartaric acid or a salt thereof, N-hydroxy iminodiacetate or a salt thereof, hydroxyethyl-ethylene diamine-tetraacetic acid or a salt thereof, 1- or 3-propanediamine tetra acetic acid or a salt thereof, 1- or 3-diamino-2-hydroxy propane tetra-acetic acid or a salt thereof, sodium gluconate, hydroxy ethane diphosphonic acid or a salt thereof, and phosphoric acid or a salt thereof.

Wetting agents, emulsifiers and lubricants, such as sodium lauryl sulfate and magnesium stearate, as well as coloring agents, release agents, coating agents, sweetening, flavoring and perfuming agents, preservatives, solubilizing agents, buffers and antioxidants can also be present in the compositions.

The solubility of compounds of formula 1 can be improved in aqueous media by the addition of solubilizing or complexing agents.

Pharmaceutically-acceptable solubilizing agents that may also be present in the compositions include, but are not limited to polyoxyethylene sorbitan fatty acid esters (including polysorbate 80), polyoxyethylene stearates, benzyl alcohol, ethyl alcohol, polyethylene glycols, propylene glycol, glycerin, cyclodextrin, and poloxamers.

Pharmaceutically-acceptable complexing agents that may be present in the compositions include, but are not limited to, cyclodextrins (alpha, beta, gamma), especially substituted beta cyclodextrins such as 2-hydroxypropyl-beta, dimethyl beta, 2-hydroxyethyl beta, 3-hydroxypropyl beta, trimethyl beta.

Pharmaceutical compositions of the present invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, chelating agents, solutes which render the formulation isotonic with the blood of the intended recipient or suspending or thickening agents. In the examples, the active ingredients are brought together with the pharmaceutically acceptable carriers in solution and then lyophilized to yield a dry powder. The dry powder is packaged in unit dosage form and then reconstituted for parental administration by adding a sterile solution, such as water or normal saline, to the powder.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms upon the compounds of the present invention may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

One formulation of a compound of the present invention contains ascorbic acid (for example, wherein the molar ratio of ascorbic acid to a compound of formula 1 is in the range from about 0.1 to about 10; or in another example, wherein the molar ratio of ascorbic acid to a compound of formula 1 is in the range from about 1 to about 6).

### Methods of Making

A variety of methodologies can be adapted for generating the compounds of the present invention. In general, the steps involve (1) converting the ansamycin to a 17-demethoxy-17-amino analog (e.g., 17-AAG), (2) reducing the benzoquinone in the ansamycin to give a hydroquinone, and (3) combining the reduced benzoquinone-containing ansamycin with at least one pharmaceutically acceptable excipient.

In certain embodiments, synthetic methodology is used to create analogs of a natural product isolated from an organism using known methods. For example, geldanamycin is isolated from a fermentation culture of an appropriate micro-organism and may be derivatized using a variety of functionalization reactions known in the art. Representative examples include metal-catalyzed coupling reactions, oxidations, reductions, reactions with nucleophiles, reactions with electrophiles, pericyclic reactions, installation of protecting groups, removal of protecting groups, and the like. Many methods are known in the art for generating analogs of the various benzoquinone ansamycins (for examples, see U.S. Pat. Nos. 4,261,989; 5,387,584; and 5,932,566 and J. Med. Chem. 1995, 38, 3806-3812, herein incorporated by reference). These analogs are readily reduced, using methods outlined below, to yield the 18,21-dihydro derivatives of the present invention.

A variety of methods and reaction conditions can be used to reduce the benzoquinone portion of the ansamycin. Sodium hydrosulfite may be used as the reducing agent. Other reducing agents that can be used include, but are not limited to, zinc dust with acetic anhydride or acetic acid, ascorbic acid and electrochemical reductions. Reduction methods are further described in WO 2005/063714.

Reduction of the benzoquinone moiety of the ansamycin derivative may be accomplished using sodium hydrosulfite in a biphasic reaction mixture. Typically, the geldanamycin analog is dissolved in an organic solvent, such as EtOAc. Other solvents that can be used include, but are not limited to, dichloromethane, chloroform, dichloroethane, chlorobenzene, THF, MeTHF, diethyl ether, diglyme, 1,2-dimethoxyethane, MTBE, THP, dioxane, 2-ethoxybutane, methyl butyl ether, methyl acetate, 2-butanone, water and mixtures thereof. Two or more equivalents of sodium hydrosulfite are then added as a solution in water (5-30% (m/v), preferably 10% (m/v)), to the reaction vessel at room temperature. Aqueous solutions of sodium hydrosulfite are unstable and therefore need to be freshly prepared just prior to use. Vigorous mixing of the biphasic mixture ensures reasonable reaction rates.

The reaction can readily be followed at this step by visual inspection since the starting material 17-AAG has a purple color which will disappear as the reaction proceeds to the product dihydro-17AAG, which is yellow. However, HPLC/UV or other analytical methods can be used to monitor the reaction.

Upon completion of the reduction, the crude reaction product may be used directly in the preparation of the pharmaceutical composition of the present invention without purification to minimize oxidation of the hydroquinone.

Methods of preparing the formulations or compositions comprise the step of contacting a compound of the present invention with a carrier and, optionally, one or more accessory ingredients. In general, the formulations are prepared by uniformly and intimately bringing into association a compound of the present invention with liquid carriers (liquid formulation), liquid carriers followed by lyophylization (powder formulation for reconstitution with sterile water or the like), or finely divided solid carriers, or both, and then, if necessary, shaping or packaging the product. Examples of liquid carriers include, but are not limited to acetonitrile, alcohols, THF, acetonem butanone, and polyols. Methods for preparing the pharmaceutical compositions of the present invention are described below.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Preparation of a Hydroquinone Analog of 17-AAG

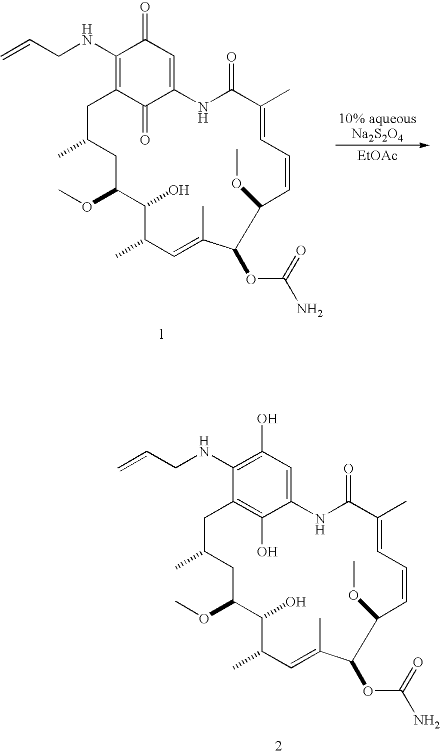

17-Allylaminogeldanamycin (1) (0.548 g, 0.937 mmol, 1.0 equiv) was dissolved in 12.0 mL ethyl acetate and stirred with an aqueous solution of sodium hydrosulfite (sodium hydrosulfite (1.2 g) in water (12 mL)). The deep purple solution turned yellow after 5 min and the mixture was stirred for an additional 25 min. The organic layer was collected, dried over MgSO4, and concentrated under reduced pressure to yield the desired hydroquinone 2 (0.500 g, 0.85 mmol, 90% yield).

### Example 2

### Preparation of a Pharmaceutical Composition of Hydroquinone 2

To the hydroquinone 2 (58 mg, 0.99 mmol) in acetonitrile (4 mL) was added a solution of ascorbic acid (0.872 g, 4.95 mmol, 5 eq) in water (4 mL). The solution was frozen and lyophilized to give an off-white powder.

### Example 3

### Preparation of a Pharmaceutical Composition of Hydroquinone 2

To the hydroquinone 2 (58 mg, 0.99 mmol, 1 eq) in t-BuOH (4 mL) was added a pH 3.1 buffered solution (4 mL; 2.4 mM, EDTA 50 mM ascorbic acid, and 50 mM citric acid) and the resulting solution was frozen and lyophilized to give a pharmaceutical composition.

### EQUIVALENTS & INCORPORATION BY REFERENCE

It is understood that the examples and embodiments described herein are for illustrative purposes only and that various modifications or changes in light thereof will be suggested to persons skilled in the art and are included within the spirit and purview of this application and scope of the appended claims. All of the U.S. patents and U.S. patent application publications cited herein are hereby incorporated by reference in their entirety for all purposes.

1. A pharmaceutical composition comprising: at least one pharmaceutically acceptable excipient; and a compound of formula 1: 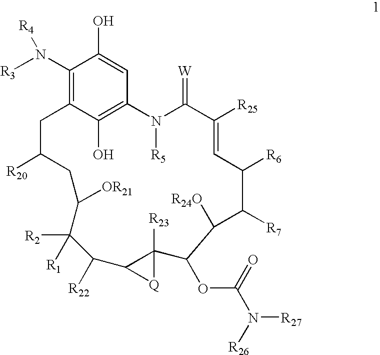
wherein independently for each occurrence:
W is oxygen or sulfur;
Q is oxygen, NR, N(acyl) or a bond;
R for each occurrence is independently selected from the group consisting of hydrogen, alkyl, cycloalkyl, heterocycloalkyl, aralkyl, heteroaryl, and heteroaralkyl;
R₁ is hydroxyl, alkoxyl, —OC(O)R₈, —OC(O)OR₉, —OC(O)NR₁₀R₁₁, —OSO₂R₁₂, —OC(O)NHSO₂NR₁₃R₁₄, —NR₁₃R₁₄, or halide; and R₂ is hydrogen, alkyl, or aralkyl; or R₁ and R₂ taken together, along with the carbon to which they are bonded, represent —(C═O)—, —(C═N—OR)—, —(C═N—NHR)—, or —(C═N—R)—;
R₃ and R₄ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆; or R₃ taken together with R₄ represent a 4-8 membered optionally substituted heterocyclic ring;
R₅ is selected from the group consisting of H, alkyl, aralkyl, and a group having the formula 1a: 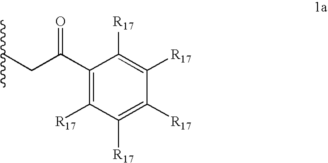
wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocyclyl;
R₆ and R₇ are both hydrogen; or R₆ and R₇ taken together form a bond;
R₈ is hydrogen, alkyl, alkenyl, alkynyl, aryl, cycloalkyl, heterocycloalkyl, aralkyl, heteroaryl, heteroaralkyl, or —[(CR₂)ₚ]—R₁₆;
R₉ is alkyl, alkenyl, alkynyl, aryl, cycloalkyl, heterocycloalkyl, aralkyl, heteroaryl, heteroaralkyl, or —[(CR₂)ₚ]—R₁₆;
R₁₀ and R₁₁ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆; or R₁₀ and R₁₁ taken together with the nitrogen to which they are bonded represent a 4-8 membered optionally substituted heterocyclic ring;
R₁₂ is alkyl, alkenyl, alkynyl, aryl, cycloalkyl, heterocycloalkyl, aralkyl, heteroaryl, heteroaralkyl, or —[(CR₂)ₚ]—R₁₆;
R₁₃ and R₁₄ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆; or R₁₃ and R₁₄ taken together with the nitrogen to which they are bonded represent a 4-8 membered optionally substituted heterocyclic ring;
R₁₆ for each occurrence is independently selected from the group consisting of hydrogen, hydroxyl, acylamino, —N(R₁₈)COR₁₉, —N(R₁₈)C(O)OR₁₉, —N(R₁₈)SO₂(R₁₉), —CON(R₁₈)(R₁₉), —OC(O)N(R₁₈)(R₁₉), —SO₂N(R₁₈)(R₁₉), —N(R₁₈)(R₁₉), —OC(O)OR₁₈, —COOR₁₈, —C(O)N(OH)(R₁₈), —OS(O)₂OR₁₈, —S(O)₂OR₁₈, —OP(O)(OR₁₈)(OR₁₉), —N(R₁₈)P(O)(OR₁₈)(OR₁₉), and —P(O)(OR₁₈)(OR₁₉);
p is 1, 2, 3, 4, 5, or 6;
R₁₈ for each occurrence is independently selected from the group consisting of hydrogen, alkyl, aryl, cycloalkyl, heterocycloalkyl, aralkyl, heteroaryl, and heteroaralkyl;
R₁₉ for each occurrence is independently selected from the group consisting of hydrogen, alkyl, aryl, cycloalkyl, heterocycloalkyl, aralkyl, heteroaryl, and heteroaralkyl; or R₁₈ taken together with R₁₉ represent a 4-8 membered optionally substituted ring;
R₂₀, R₂₁, R₂₂, R₂₄, and R₂₅, for each occurrence are independently alkyl;
R₂₃ is alkyl, —CH₂OH, —CHO, —COOR₁₈, or —CH(OR₁₈)₂;
R₂₆ and R₂₇ for each occurrence are independently selected from the group consisting of hydrogen, alkyl, aryl, cycloalkyl, heterocycloalkyl, aralkyl, heteroaryl, and heteroaralkyl;
provided that when R₁ is hydroxyl, R₂ is hydrogen, R₆ and R₇ taken together form a double bond, R₂₀ is methyl, R₂₁ is methyl, R₂₂ is methyl, R₂₃ is methyl, R₂₄ is methyl, R₂₅ is methyl, R₂₆ is hydrogen, R₂₇ is hydrogen, Q is a bond, and W is oxygen; R₃ and R₄ are not both hydrogen nor when taken together represent an unsubstituted azetidine; and
the absolute stereochemistry at a stereogenic center of formula 1 may be R or S or a mixture thereof and the stereochemistry of a double bond may be E or Z or a mixture thereof.

2. The pharmaceutical composition of claim 1, wherein the compound of formula 1 is selected from the group consisting of:

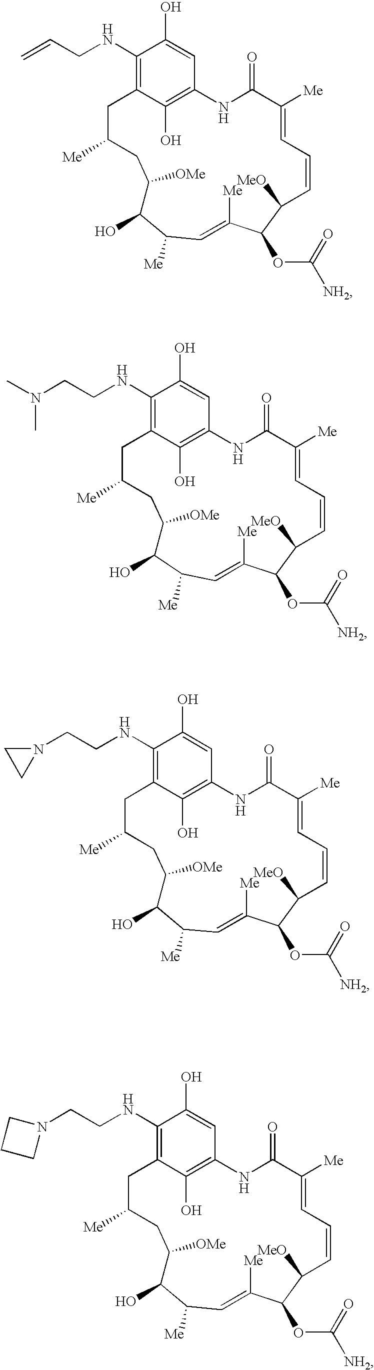

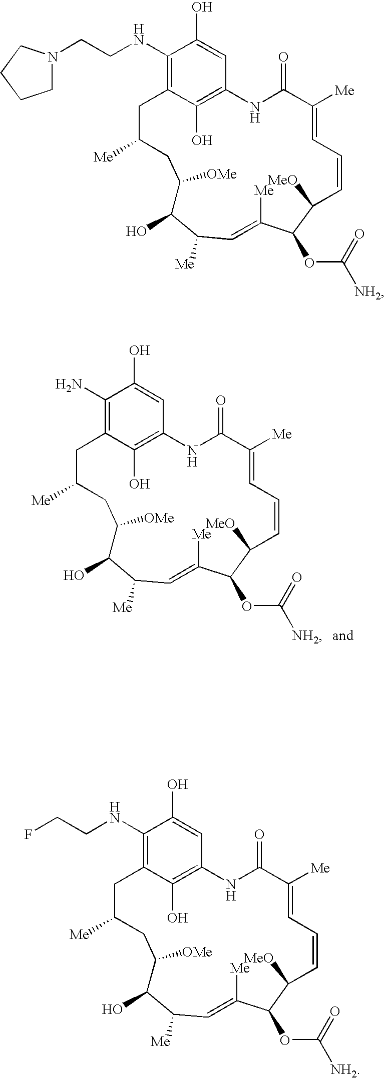

3. The pharmaceutical composition of claim 1, further comprising an antioxidant.

4. The pharmaceutical composition of claim 1, further comprising a metal chelator.

5. The pharmaceutical composition of claim 1, further comprising an antioxidant and a metal chelator.

6. The pharmaceutical composition of claim 5, wherein said antioxidant is ascorbate, cysteine hydrochloride, sodium bisulfate, sodium metabisulfite, sodium sulfite, thioglycerol, sodium mercaptoacetate, sodium formaldehyde sulfoxylate, ascorbyl palmitate, butylated hydroxyanisole, butylated hydroxytoluene, lecithin, propyl gallate, or alpha-tocopherol.

7. The pharmaceutical composition of claim 5, wherein said metal chelator is citric acid, ethylenediamine tetraacetic acid (EDTA) or a salt thereof, DTPA (diethylene-triamine-penta-acetic acid) or a salt thereof, EGTA or a salt thereof, NTA (nitriloacetic acid) or a salt thereof, sorbitol or a salt thereof, tartaric acid or a salt thereof, N-hydroxy iminodiacetate or a salt thereof, hydroxyethyl-ethylene diamine-tetraacetic acid, 1-propanediamine tetra acetic acid or a salt thereof, 3-propanediamine tetra acetic acid or a salt thereof, 1-diamino-2-hydroxy propane tetra-acetic acid or a salt thereof, 3-diamino-2-hydroxy propane tetra-acetic acid or a salt thereof, sodium gluconate, hydroxy ethane diphosphonic acid or a salt thereof, or phosphoric acid or a salt thereof.

8. The pharmaceutical composition of claim 5, wherein said antioxidant is ascorbate.

9. The pharmaceutical composition of claim 5, wherein said metal chelator is citric acid, or ethylenediamine tetraacetic acid (EDTA) or a salt thereof.

10. The pharmaceutical composition of claim 2, further comprising an antioxidant.

11. The pharmaceutical composition of claim 2, further comprising a metal chelator.

12. The pharmaceutical composition of claim 2, further comprising an antioxidant and a metal chelator.

13. The pharmaceutical composition of claim 12, wherein said antioxidant is ascorbate, cysteine hydrochloride, sodium bisulfate, sodium metabisulfite, sodium sulfite, thioglycerol, sodium mercaptoacetate, sodium formaldehyde sulfoxylate, ascorbyl palmitate, butylated hydroxyanisole, butylated hydroxytoluene, lecithin, propyl gallate, or alpha-tocopherol.

14. The pharmaceutical composition of claim 12, wherein said metal chelator is citric acid, ethylenediamine tetraacetic acid (EDTA) or a salt thereof, DTPA (diethylene-triamine-penta-acetic acid) or a salt thereof, EGTA or a salt thereof, NTA (nitriloacetic acid) or a salt thereof, sorbitol or a salt thereof, tartaric acid or a salt thereof, N-hydroxy iminodiacetate or a salt thereof, hydroxyethyl-ethylene diamine-tetraacetic acid, 1-propanediamine tetra acetic acid or a salt thereof, 3-propanediamine tetra acetic acid or a salt thereof, 1-diamino-2-hydroxy propane tetra-acetic acid or a salt thereof, 3-diamino-2-hydroxy propane tetra-acetic acid or a salt thereof, sodium gluconate, hydroxy ethane diphosphonic acid or a salt thereof, or phosphoric acid or a salt thereof.

15. The pharmaceutical composition of claim 12, wherein said antioxidant is ascorbate.

16. The pharmaceutical composition of claim 12, wherein said metal chelator is citric acid, or ethylenediamine tetraacetic acid (EDTA) or a salt thereof.
